(19) United States
(12) Patent Application Publication
(10) Pub. No.: US 2024/0324903 A1
(43) Pub. Date: Oct. 3, 2024

(54) INTRA-BODY DEVICE WITH CAPACITANCE SENSOR AND DETECTION OF ITS INTRA-BODY PLACEMENT

(71) Applicant: Sword Health, S.A., Porto (PT)

(72) Inventors: Daipan Lee, Draper, UT (US); Gustavo Seixas de Sá Burmester, Porto (PT); Pedro Fillipe Xavier Rodrigues, Porto (PT); José Carlos Coelho Alves, Porto (PT); Márcio Filipe Moutinho Colunas, Porto (PT); João Paulo Dias Andrade, Porto (PT); Marta Maria Cardeano Pedrosa e Milheiro Maia, Porto (PT); Virgílio António Ferro Bento, Porto (PT)

(21) Appl. No.: 18/618,885

(22) Filed: Mar. 27, 2024

(30) Foreign Application Priority Data
Mar. 30, 2023 (EP) 23398007.7

(51) Int. Cl. A61B 5/11; A61B 5/00; A61B 5/06

(52) U.S. Cl. CPC A61B 5/1107; A61B 5/061; A61B 5/6867; A61B 2505/09; A61B 2560/0228; A61B 2560/0406; A61B 2560/0468; A61B 2562/0247; A61B 2562/046

(57) ABSTRACT

Examples described herein relate to an intra-body device and to detection of its intra-body placement. The intra-body device may form part of a pelvic floor muscle tracking system. Capacitance measurements are taken by a capacitance sensor of the intra-body device. The capacitance sensor is arranged such that it extends along at least part of a length of the intra-body device. The capacitance measurements are processed to determine positioning of the intra-body device in relation to a cavity of a body of a user. The cavity may be a vagina or an anus of the user. Operation of the pelvic floor muscle tracking system may be controlled based on the determined positioning.

### CLAIM OF PRIORITY

This application claims the benefit of priority to European Patent Application No. 23398007.7, filed on Mar. 30, 2023, which is incorporated herein by reference in its entirety.

### TECHNICAL FIELD

The present disclosure relates to the field of intra-body devices. More particularly, but not exclusively, the disclosure relates to the incorporation of capacitance sensors into intra-body devices, and the monitoring of the status of such intra-body devices.

### BACKGROUND

Pelvic floor muscle exercises can lead to significant health benefits, such as reducing the risk of pelvic floor dysfunction or disorders, both in female and male users. A user can perform pelvic floor muscle exercises as a preventative measure or, where the user is already experiencing a pelvic floor issue, as part of a recovery or rehabilitation program.

In this context, it may be desirable to perform physical exercising or rehabilitation under supervision, such as the supervision of a personal trainer or a physical therapist that observes and monitors the activity of the user, and prescribes certain movements or exercises to the user. Alternatively, or in addition to the aforementioned supervision, physical exercising or rehabilitation programs may utilize data provided by devices or systems, such as sensors, motion tracking systems, or pressure tracking systems. The data provided by such devices or systems can enable more detailed monitoring, since certain parts of the body cannot be manually inspected or monitored at a level of detail that would be convenient, desirable, or scalable.

### BRIEF DESCRIPTION OF THE DRAWINGS

To complete the description and in order to provide for a better understanding of the disclosure, a set of drawings is provided. The drawings form an integral part of the description and illustrate examples of the disclosure, which should not be interpreted as restricting the scope of the disclosure, but just as examples of how the disclosure can be carried out. The drawings comprise the following figures:

FIG. 1 is a perspective view of an intra-body device, according to some examples.

FIG. 2 is a block diagram illustrating a pelvic floor muscle tracking system, according to some examples.

FIG. 3 is a diagrammatic illustration of a capacitance sensor for intra-body devices, according to some examples.

FIG. 4 is a diagrammatic illustration of a capacitance sensor for intra-body devices, according to some examples.

FIG. 5 is a diagrammatic illustration of an intra-body device and a cavity of a user, according to some examples.

FIG. 6A is a diagrammatic illustration of an intra-body device and a cavity of a user, according to some examples, wherein a position of the intra-body device at a first point in time is shown.

FIG. 6B is a diagrammatic illustration of the intra-body device and the cavity of FIG. 6A, according to some examples, wherein the position of the intra-body device at a second point in time is shown.

FIG. 7 is a table of a predetermined set of calibration values, according to some examples.

### DETAILED DESCRIPTION

An intra-body device can be used to obtain data relating to a user's physical exercising or rehabilitation, such as the user's performance of pelvic floor muscle exercises. An intra-body device can be insertable into a cavity, such as the vagina or anus of the user, and may include one or more sensors for measuring the forces or pressure that muscles of the pelvic floor exert on the intra-body device while inserted in the cavity.

The pressure applied by the muscles of the pelvic floor is not observable by a personal trainer or a physical therapist, but pressure data from this region can be indicative of the health condition of the pelvic floor and the manner in which pelvic floor exercises are performed, making such an intra-body device a useful apparatus. The obtaining and digital processing of data provided by these devices has made it possible to supervise the rehabilitation or exercises of the user digitally and in a fully or partially automated manner (e.g., without physical or real-time monitoring by a trainer or a therapist).

However, in many scenarios, it is important that the intra-body device is properly positioned in the cavity of the user, for example, to ensure that adequate measurements are taken and/or that the measurements will be sufficiently informative. Furthermore, even when the intra-body device is properly positioned, the measurements may not accurately represent the activity of the pelvic floor if the intra-body device is not calibrated to the actual position thereof among a range of positions within the cavity. Examples described herein provide a technical solution that is capable of addressing or alleviating at least some of these technical problems.

A first aspect relates to a method. The method may include digitally processing capacitance measurements taken by at least one capacitance sensor of an intra-body device, and digitally determining whether the intra-body device is at least partially introduced in a cavity of a body of a user or not introduced in the cavity based on the processed capacitance measurements and one or more predetermined thresholds. In some examples, the at least one capacitance sensor is arranged on the intra-body device such that the at least one capacitance sensor extends along at least part of a length of the intra-body device.

The cavity may be a vagina or an anus of the user. During operation, the cavity receives the intra-body device when the user is to use the intra-body device, for example, for exercising, rehabilitation, or pressure measuring. Such activities may be performed in a supervised manner. The measurements provided by the at least one capacitance sensor before, during, or after the intra-body device is placed in the cavity can be processed for the determination of the introduction or insertion status of the intra-body device.

In some examples, the digital processing of the capacitance measurements, which are taken over time with a predetermined frequency of the sensor and, thus, show their evolution, is such that one or more measured values are to be considered.

For instance, a single measured value at a plurality of points of the at least one capacitance sensor, or a single measured value per sensor of a plurality of sensors of the at least one capacitance sensor can be processed to determine whether the intra-body device is within the cavity or not. The magnitude of the measured values can be compared with one or more predetermined thresholds or with one or more measured values of one or more other sensors of the at least one capacitance sensor. A measurement of the type of, for example, a complex number can be directly compared or be first mathematically (digitally) processed before comparison with one or more predetermined thresholds or measured values of other points or sensors of the at least one capacitance sensor.

In some examples, a plurality of measured values can be processed to determine whether the intra-body device is within the cavity or not. In this regard, either a plurality of measured values per point or per sensor may be processed. The processing may thus consider any changes there may be in the measured values over time to determine the introduction or insertion status of the intra-body device, meaning that a significant change in measured capacitance in a short time span (e.g., 500 milliseconds, 1 second, 2 seconds, or 5 seconds) is indicative of a change in the introduction or insertion status. Depending on the values before and after the sudden change, the processing determines that the intra-body device has been introduced or extracted from the cavity.

In some examples, as the at least one capacitance sensor extends along at least part of a length of the intra-body device, incomplete (e.g., partial) introduction of the intra-body device can be inferred by processing measurements from at least opposite ends of the at least one capacitance sensor. The at least one capacitance sensor may extend along a major length of the device, which is the length or axis that is parallel to the canal or cavity where the intra-body device is introduced. The length of the at least one capacitance sensor may be attained by having different sensors of the at least one capacitance sensor, that is to say, there are two or more sensors arranged along the length dimension; alternatively, it may be attained by having one or more capacitance sensors that are capable of measuring the capacitance at different parts of the length of the intra-body device like.

The capacitance sensor(s) may have a surface contacting or almost contacting (namely, there is a gap) a wall of the cavity, such as a vaginal wall, and/or a fluid, such as a fluid of the body of the user or a lubricant.

A method as described herein may be a computer-implemented method that runs in one or more processing devices. The method may be run in an isolated manner and/or in a distributed manner. That is to say, one, some or all steps or operations may be run by a same processing device, or one or some steps may be run by one processing device and some other step or steps may be run by one or more other processing devices or even be run in distributed manner, which means that several processing devices cooperate to run one or more steps. In this sense, the steps are digitally run. The processing device or devices may be included in the intra-body device itself, be external to the intra-body device, or a combination thereof. For example, a processing device such as, but without limitation, a mobile phone, a tablet, a personal computer, a server, a field-programmable gate array, or an application specific integrated circuit, can be utilized, and may be remote from the intra-body device.

A method as described herein may be performed by a pelvic floor muscle tracking system, motion tracking system, or part thereof.

A second aspect relates to a method. The method may include digitally processing capacitance measurements taken by at least one capacitance sensor of an intra-body device, and digitally providing a pelvic floor motion tracking sequence of how muscles of a pelvic floor of a user are contracting or elongating by digitally determining whether the intra-body device moves inwards or outwards in a cavity (e.g., vagina or anus) of the user, based on the processed capacitance measurements. In some examples, the at least one capacitance sensor is arranged on the intra-body device such that the at least one capacitance sensor extends along at least part of a length of the intra-body device.

In some examples, the processed measurements of the at least one capacitance sensor, while the intra-body device is at least partially inside the cavity, are used in the provision of the pelvic floor motion tracking sequence. Therefore, the method generates information about the activity of the user in terms of contraction and elongation of the muscles.

A third aspect relates to a method. The method may include digitally processing capacitance measurements taken by at least one capacitance sensor of an intra-body device, and digitally determining whether at least a first portion of the intra-body device is contacting one or more walls delimiting a cavity (e.g., vagina or anus) of a user based on the processed capacitance measurements. In some examples, the at least one capacitance sensor is arranged on the intra-body device such that the at least one capacitance sensor extends along at least part of a length of the intra-body device. The first portion may encompass a part or surface of the intra-body device where the at least one capacitance sensor is arranged.

In some examples, the method detects adequate positioning of the intra-body device within the cavity. For example, the method includes detecting whether the first portion is completely contacting one or more walls inside the cavity. When this does not occur, a system (e.g., pelvic floor muscle tracking system) may determine that the positioning of the intra-body device is considered incorrect or inadequate.

In some examples of the second and/or third aspects, the method further comprises digitally determining whether the intra-body device is at least partially introduced in a cavity of a body of a user or not introduced in the cavity based on the processed capacitance measurements and one or more predetermined thresholds.

In some examples of the first, second and/or third aspects, digitally determining whether the intra-body device is at least partially introduced in the cavity comprises determining a level of introduction or insertion of the intra-body device within the cavity.

In some examples, the processing previously described also enables estimation of how far or to what extent the intra-body device is introduced within the cavity, thereby quantifying which portion of the intra-body device is within the cavity and which portion is outside the cavity when the intra-body is not fully introduced nor fully outside the cavity.

Knowing whether part of the intra-body device is not within the cavity, and how long that part is, may provide technical advantages in the system. For example, when the user has limited sensory processing and, thus, cannot tell whether the device has been fully introduced in the cavity, or partially introduced, and to what extent, the system may utilize this information to facilitate operation of the intra-body device by the user. The same kind of information may also be useful during exercising or rehabilitation sessions as the user may not advert or report that the intra-body device has moved and has partially slipped out from the cavity, or more or less part of the intra-body device is now outside the cavity while initially only a specific part was partially out. This situation may occur as the muscles within the cavity elongate, contract and/or change their shape in general due to the pressure applied by the user to the pelvic floor muscles, and due to the motion of the body members.

When the activity of the user is tracked by the system (e.g., pelvic floor muscle tracking system) for supervision thereof, inadequate level of introduction of the intra-body device may lead to limited or flawed supervision that may eventually result in an injury or poor user experience or recovery. In some cases, inadequate level of introduction means that too much of the intra-body device (e.g., more than 5%, 10%, 20% or more of the length of the intra-body device) is outside the cavity, or that the device is not fully inserted in the cavity.

In the context of the present disclosure, full introduction may include complete or substantially complete introduction of at least a main body of the intra-body device, which is a part of the device that, in many examples, embeds electronics such as sensors and a battery. In addition to the main body, the intra-body device can include a member, e.g., a protruding arm or a protruding flap, not meant to be introduced in the cavity as it is the member to be pulled for extraction of the intra-body device from the cavity. The member not introducible in the cavity may also include a data communications module. Hence, it will be appreciated that full introduction does not mean the introduction of such member, but rather complete or substantially complete introduction of the part of the intra-body device that is designed to be inserted into the cavity.

In some examples of the first and/or second aspects, the method further includes digitally determining whether at least a first portion of the intra-body device is contacting one or more walls delimiting the cavity based on the processed capacitance measurements. The first portion encompasses a part or surface of the intra-body device where the at least one capacitance sensor is arranged.

When the intra-body device is contacting walls of, for example, the vaginal canal or the anal canal, measurements of the at least one capacitance sensor may be within a predetermined range of values. The determination can thus be made by comparing the capacitance measurements with the predetermined range of values. In some examples, when the measurements corresponding to one or several parts of the first portion are out of range, the determination is that at least part of the first portion is not in contact with the walls.

Additionally, or alternatively, comparing capacitance measurements in pairs, e.g, one to each other, provides the differences in values between several parts of the first portion. A difference between a pair of measurements exceeding a predetermined threshold may indicate that the sensor at a first position contacts the wall whereas the sensor at a second position does not contact the wall (or that a first sensor of the at least one capacitance sensor contacts the wall whereas a second sensor of the at least one capacitance sensor does not).

Uneven contact between the intra-body device, at least of the first portion thereof, and the walls inside the cavity may correspond to an incorrect positioning of the device. This situation can be detected by the method, and the user can be accordingly notified by way of an apparatus for notification so that corrective action may be taken. The apparatus for notification may form part of a system as described herein.

The intra-body device may include the at least one capacitance sensor and at least one pressure sensor. In some examples of the first, second, and/or third aspects, the method further includes digitally processing exercise measurements taken by at least one pressure sensor of the intra-body device, with said exercise measurements being taken when the user is doing physical exercise with the intra-body device introduced in the cavity. The method may include digitally providing pelvic floor muscle tracking of how muscles of a pelvic floor of the user are contracting or elongating based on the processed exercise measurements. The method may further include calibrating the pelvic floor muscle tracking by using a predetermined set of calibration values or a predetermined model with a set of calibration values based on the determined level of introduction of the intra-body device and/or based on a determination of whether at least the first portion of the intra-body device is contacting one or more walls delimiting the cavity, with the predetermined set or model relating a predetermined level of introduction of the intra-body device to a predetermined pressure value.

Even though the measurements of the at least one pressure sensor may be accurate and, thus, the pressure exerted by the muscles in the pelvic floor is correctly trackable and monitorable, they may fail to not account properly for the positioning of the intra-body device within the cavity. For example, the pressure measurements are depended on the location of the intra-body device inside the cavity because for a same exerted pressure, the different muscles and portions thereof behave differently in terms of their elongation, contraction and reshaping, hence the pressure is different at different locations of the pelvic floor.

To correctly establish what actions, movements, or exercises the user is doing, it can be important for pressure measurements to be correlated to where the intra-body device is within the cavity. For example, the pressure measurements should be related to where along the cavity the device is, including, for example, full introduction, or how part of the device is contacting walls of the cavity. In some examples, this is made possible by a system that executes a method as described herein, using the processed capacitance measurements. The measurements are indicative of any of these two situations, and can be used to perform a calibration of the pelvic floor muscle tracking. To this end, the predetermined set of calibration values or model can be defined in terms of the device's position inside the cavity, such as the level of introduction, and whenever calibration is to be conducted, upon determining the device's position (e.g., how the first portion is contacting walls or not, or the level of introduction) the corresponding calibration values can be considered to adjust the pelvic floor muscle tracking. By way of example, when the determined level of introduction is 97%, the calibration values at or closest to such level of introduction are applied or considered for scaling the exercise measurements accordingly, while when the determined level of introduction is 90%, the calibration values at or closest to such level of introduction are applied or considered for scaling the exercise measurements accordingly, and so on.

In some examples of the first, second, and/or third aspects, the method further includes digitally processing calibration measurements taken by at least one pressure sensor of the intra-body device, with said calibration measurements being taken when the user is conducting a calibration of the intra-body device with the intra-body device introduced in the cavity. The method may include digitally providing the predetermined set of calibration values or the model such that:
the set or the model relates each determined level of introduction of the intra-body device to at least one pressure value of the processed calibration measurements taken at the determined level of introduction; or
the set or the model relates each determined contact of the first portion of the intra-body device with the one or more walls delimiting the cavity to at least one pressure value of the processed calibration measurements taken when it is determined that there is the contact of the first portion of the intra-body device with the one or more walls delimiting the cavity.

In some examples, the provision of the predetermined set of calibration values or model is enabled by the user conducting a calibration procedure whereby the resulting pressure measurements are related to one or more positions of the intra-body device within the cavity. The provision of the set or model, and/or the incorporation of additional calibration values to an existing set or model, can be triggered in some cases whenever the determined position of the intra-body device changes and the new determined position is inside a range of valid positions within the cavity, thereby providing calibrating values on-the-fly.

By relating the pressure measurements to the levels of introduction or contacts of the device (first portion thereof) with the walls, the set of calibration values and/or the model can be produced for subsequent calibration.

In some examples of the first, second, and/or third aspects, the at least one pressure value for the provision of the predetermined set of calibration values or model comprises a maximum pressure exerted by the muscles of the pelvic floor and/or a minimum pressure exerted by the muscles of the pelvic floor.

In some examples, for a more accurate calibration of the pelvic floor muscle tracking, the pressure value(s) during the provision of a set of calibration values or model involves a maximum pressure and/or minimum pressure. The resulting calibration values or model then have an absolute value for comparison or scaling of the subsequent pressure measurements during exercise or rehabilitation sessions.

In some examples of the first, second, and/or third aspects, the method further includes digitally commanding to provide or providing one or more user perceptible signals indicative of the calibration of the intra-body device to be conducted by the user. In other words, the system (e.g., the pelvic floor muscle tracking system) may provide an instruction to the user to perform one or more calibration operations.

Guidance may be provided to the user via the system to conduct the calibration procedure within some predetermined parameters that improve the accuracy of the calibration of the pelvic floor muscle tracking.

In some examples of the first, second, and/or third aspects, the method further includes digitally adjusting, based on the determined introduction of the intra-body device and/or based on the determination of whether at least the first portion of the intra-body device is contacting one or more walls delimiting the cavity, operation of the intra-body device or operation of a pelvic floor muscle tracking system comprising the intra-body device. The intra-body device or the pelvic floor muscle tracking system may thus be controlled or adjusted based at least in part on the determined introduction level or whether at least the first portion of the intra-body device is contacting one or more walls delimiting the cavity.

In some examples, capacitance measurements taken by the at least one capacitance sensor of an intra-body device are processed to determine positioning of the intra-body device in relation to the cavity, and the operation of the system (e.g., the pelvic floor muscle tracking system) is then controlled based on the determined positioning.

The intra-body device or another part of the system may be controlled to adapt the respective operation to the status of the intra-body device with respect to its position (including orientation of the device relative to the cavity) within the cavity. In this sense, in some examples, digitally adjusting or controlling the operation comprises, when it is determined that the intra-body device is not introduced in the cavity or is only partially introduced therein or is fully introduced therein but with a position outside a predetermined range of valid positions (including predetermined range of valid orientations), commanding or causing activation of an apparatus for notifying the user of the partial introduction or non-introduction of the intra-body device. The intra-body device or the pelvic floor muscle tracking system may include the apparatus that notifies the user, and, in the case of the latter, the apparatus may be remote from the intra-body device and may additionally be remote from the user.

Different ways of notifying the user are possible within the scope of the present disclosure. For example, but without limitation, the apparatus may comprise a sound-emitting device and/or a vibrator and/or a light-emitting device and/or a display. By way of example, when the intra-body device slips out from the cavity, a screen providing guidance instructions to the user about the performance of some exercises may show an alert, and/or some loudspeakers produce a sound. A user perceptible signal may thus be or cause various types of notifications.

In some examples of the first and/or third aspects, the method further includes digitally providing a pelvic floor motion tracking sequence of how muscles of a pelvic floor of the user are contracting or elongating by digitally determining whether the intra-body device moves inwards or outwards in the cavity based on the processed capacitance measurements. The method may include controlling operation of the system (e.g., the pelvic floor muscle tracking system) by providing the pelvic floor motion tracking sequence.

Time evolution of the capacitance measurements, upon processing the measurements, may be indicative of the behavior of the intra-body device with respect to movement relative to the cavity. For example, inwards motion, which is the motion in the direction of introduction of the device, and the outwards motion, which is the motion in the direction of extraction or slipping out of the device, can be related to the contraction and elongation of the muscles of the pelvic floor. In some cases, the inwards motion can be linked to the contraction, and the outwards motion can be linked to the elongation, and in some other cases it is the other way around.

In some examples, the pelvic floor motion tracking sequence comprises a data stream, which can take any form or representation known in the art, that includes information about the contraction and elongation of the muscles in the pelvic floor versus time. The sequence may show how much the muscles have contracted and elongated in each repetition of an exercise, and/or for how long they have stayed that way, and/or how many times any of these actions have occurred. The sequence may be comparable with a predetermined pelvic floor muscle exercising or rehabilitation plan to establish how close the user has followed the plan, or whether there has been some improvement or worsening over time (for example, in the course of several days, several weeks, several months, etc.) in the condition of the pelvic floor muscles, etc.

The provision of the sequence may occur at any point in time, for example: while the user is doing physical exercise with the intra-body device introduced in the cavity (e.g., on-the-fly), after the user has done the physical exercise with the intra-body device introduced in the cavity, or during or after the user is conducting a calibration procedure so that the calibration can be checked.

Further, in some examples, the determined inwards or outwards motion is associated with an incorrect position or introduction of the intra-body device in the cavity. In some examples, the method includes digitally registering the incorrect position or introduction.

Various situations exist in which the intra-body device might be expected to be located in the cavity in a certain manner, and specific inwards or outwards motion would not be compatible with such location. In some examples, the method causes the system to operate such that, whenever such inwards or outwards motion is determined to exist, the positioning of the intra-body device can be flagged as being incorrect for subsequent corrective action to be taken.

In some examples of the first, second, and/or third aspects, providing the pelvic floor motion tracking sequence is based on both the digital determination of whether the intra-body device moves inwards or outwards in the cavity based on the processed capacitance measurements, and exercise measurements taken by at least one pressure sensor of the intra-body device, with said exercise measurements being taken when the user is doing physical exercise with the intra-body device introduced in the cavity.

In some examples, the inwards and outwards motion data and the exercise measurements are processed in a combined manner whereby the values of one stream of data can build upon the values of the other stream of data. For example, the system can automatically apply the inwards/outwards motion to increase the confidence in the conclusions extracted from the exercise measurements, and vice versa.

Diverse ways of combining the two sources of data are possible. For example, the processing of the two can be performed by the system relative to a set of thresholds that define the confidence in the provided pelvic floor motion tracking sequence, such as a truth table, a rule-based model, or a decision tree model.

In some examples of the first, second, and/or third aspects, the method further includes repeating the steps/operations of digitally processing the capacitance measurements and digitally determining whether the intra-body device is at least partially introduced in the cavity a plurality of times, and in each repetition using at least one new capacitance measurement taken by the at least one capacitance sensor when the user is doing the physical exercise.

Digitally adjusting or controlling the operation of the system may include, when it is determined that the intra-body device is in one or some of the plurality of times/repetitions not introduced in the cavity or is only partially introduced therein, halting the provision of pelvic floor muscle tracking at least until it is determined that the intra-body device is introduced in the cavity again. The same type of looped processing can be conducted by the system to require full introduction of the intra-body device in the cavity, or a first portion of the intra-body device contacting wall or walls of the cavity within a predetermined range of acceptable capacitance measurement values.

In some examples, the user can make sure that the insertion of the intra-body device is correct throughout an exercising or rehabilitation session by ensuring continued, automated tracking and supervision of the insertion status. For example, the system, by repeating the automated intra-body device insertion checks during the entire exercising or rehabilitation session, the pelvic floor muscle tracking can be automatically halted by the system when the level of introduction of the intra-body device becomes inadequate (e.g., the level of introduction is below a predetermined introduction value). In some examples, additionally to the halting, the method includes commanding or causing activation of an apparatus for notifying the user of the partial introduction or non-introduction of the intra-body device so that the user can review the introduction of the intra-body device. In addition to the halting, and as indicated before, the provision of new or additional calibration values can be triggered whenever the position of the intra-body device resulted in the halting is still within a range of valid positions within the cavity.

In some examples of the first, second, and/or third aspects, the intra-body device is a device according to a fourth aspect, as described below.

In some examples of the first, second, and/or third aspects, the intra-body device is part of a pelvic floor muscle tracking system according to a seventh aspect, as described below.

In some examples of the first, second, and/or third aspects, the method further includes arranging the intra-body device inside the cavity or causing arrangement of the intra-body device inside the cavity.

The fourth aspect relates to an intra-body device. The intra-body device includes at least one pressure sensor and/or at least one capacitance sensor. The at least one capacitance sensor may extend along at least part of a length of the intra-body device. The intra-body device includes a sensor (e.g., the at least one pressure sensor) capable of measuring the activity of muscles of the pelvic floor so that supervision (e.g., automated supervision via a pelvic floor muscle tracking system) of the exercising or rehabilitation thereof is made possible.

The at least one capacitance sensor may be arranged and/or configured to enable at least one of the following capabilities:
detecting and/or monitoring of the placement or positioning of the intra-body device in relation to the cavity (e.g., whether introduced, partially introduced, or not introduced in a cavity of the user, or whether engaging a wall);
providing of a pelvic floor motion tracking sequence; or
detecting and/or monitoring of adequate placement or positioning of the intra-body device when introduced in the cavity.

At least some of the capabilities may be implemented by the intra-body device itself, in which case the device also includes at least one processor and at least one memory for running steps of a method such as those described with reference to the aspects above.

At least some of the capabilities may be implemented by other devices that communicate with the intra-body device (directly or indirectly) and use the measurements of the latter. To this end, the intra-body device may include a data communications module/component, such as a wireless communications module/component, that allows the intra-body device to transmit data packets with the measurements of the sensors, either already processed or not, and/or to receive data packets, for instance for configuring the intra-body device.

In some examples, the at least one capacitance sensor comprises a plurality of sensing positions in, for example a linear or matrix arrangement, and/or the at least one capacitance sensor comprises a plurality of capacitance sensors in, for example, a linear or matrix arrangement.

In some examples, the linear or matrix arrangement of the sensors or sensing positions (e.g., a plurality of electrodes or a plurality of capacitors) allows the detection of the introduction status of the intra-body device at several positions thereof.

In some examples, the at least one capacitance sensor is arranged such that at least a portion thereof is on or near a first end of the intra-body device, with the first end being opposite a second end, and the second end being an introduction end of the intra-body device in a cavity of a user.

With the arrangement of the at least one capacitance sensor at the first end, it may be possible to obtain an accurate determination of the level of introduction of the intra-body device in the cavity. For example, an insertion of the device that is almost, but not completely, a full insertion thereof, will result in part of the capacitance sensor not being inside the cavity. This enables the system to accurately determine or detect the level of introduction.

In some examples, the intra-body device further comprises at least one motion sensor.

A fifth aspect relates to a computing device. The computing device may include at least one processor and at least one memory. Further, the at least one memory is configured, together with the at least one processor, to cause the computing device to carry out operations of a method as described in any of the above aspects.

In some examples, an intra-body device includes the computing device. In some examples, a pelvic floor muscle tracking system or motion tracking system includes the computing device.

A sixth aspect relates to a data processing device. The data processing device may include means or modules for carrying out operations of a method as described in any of the above aspects.

In some examples, an intra-body device includes the data processing device. In some examples, a pelvic floor muscle tracking system or motion tracking system includes the data processing device.

In some examples of the fifth and sixth aspects, the intra-body device is a device according to the fourth aspect.

The seventh aspect relates to a pelvic floor muscle tracking system. The pelvic floor muscle tracking system may include an intra-body device according to the fourth aspect. The pelvic floor muscle tracking system may include a computing device according to the fifth aspect and/or a data processing device according to the sixth aspect.

An eighth aspect relates to a computer program comprising instructions which, when the program is executed by at least one computing apparatus, cause the at least one computing apparatus to carry out operations of a method as described in any of the above aspects.

A ninth aspect relates to a computer-readable non-transitory storage medium comprising instructions which, when executed by at least one computing apparatus, cause the at least one computing apparatus to carry out operations of a method as described in any of the above aspects.

A tenth aspect relates to a data carrier signal carrying a computer program as described in the eighth aspect.

FIG. 1 shows an intra-body device 10, according to some examples. The intra-body device 10 can, for example, be an intra-vaginal device or an intra-anal device.

The intra-body device 10 (also referred to hereinafter simply as “the device 10”) is shown to include a body portion in the example form of a main body 16 (extending the length of the double-arrowed line) and a protruding member 17 for the extraction of the device 10 when inserted in a cavity. The device 10 includes at least one pressure sensor and, optionally, at least one motion sensor (not illustrated since the sensor/s is within the device 10, such as in the main body 16).

The device 10 can also include a wireless communications module, at least one memory unit (not illustrated since they are also within the device 10 in these examples), and at least one capacitance sensor 20. The at least one memory unit stores measurements of the sensors of the device 10 for processing and/or transmission, and may also store computer program code with instructions in case the intra-body device 10 is to conduct digital processing, such as processing of the measurements.

The at least one capacitance sensor 20 may be arranged externally, such as on the surface of the device 10 as shown in FIG. 1 (e.g., the outer surface). However, the capacitance sensor 20 may also be within the device 10. The at least one capacitance sensor 20 can be arranged such that a second end 22 thereof is at or near, or reaches a second end 12 of the device 10 (or at least the second end 12 of the main body 16 thereof). A first end of the sensor 20 may be at or near, or reach a first end 11 of the device 10 (or at least the first end 11 of the main body 16 thereof), but it can also be somewhere else along the length of the intra-body device 10, such as in the examples of FIG. 1. The first end 11 of the device 10, which is opposite the second end 12, corresponds to the end that is first introduced into the cavity of a user.

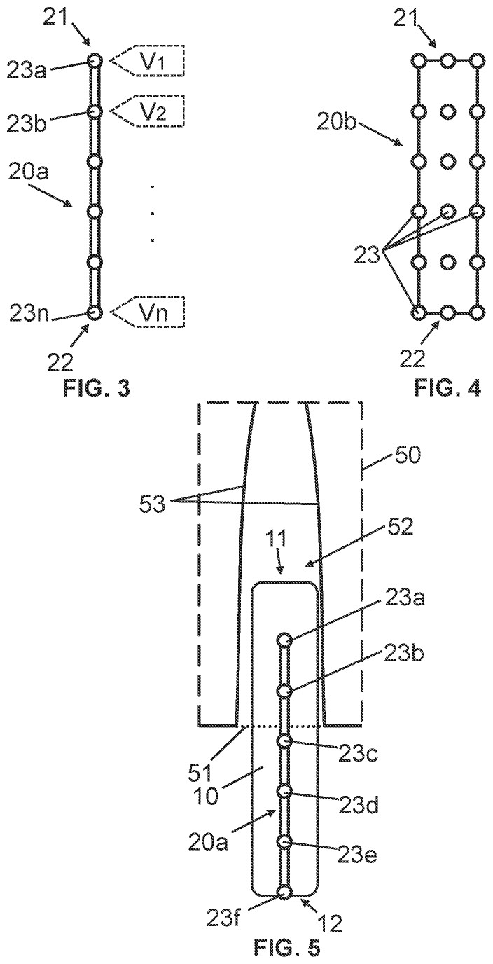

As shown, for example, in FIG. 3 and FIG. 4, the at least one capacitance sensor 20 may be arranged in linear form, such as a projection of the sensor 20 on a plane is such that it substantially extends in one dimension, or in matrix form, such as a projection of the sensor 20 on a plane is such that it substantially extends in two dimensions.

In some examples, the at least one capacitance sensor 20 is a single sensor that is capable of measuring capacitance at multiple positions thereof throughout an area or surface of the sensor 20 (e.g., along its length).

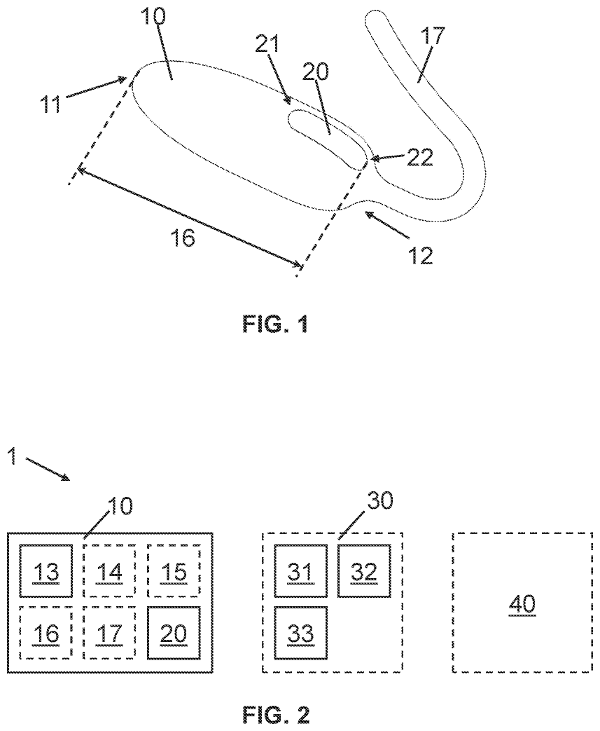

FIG. 2 shows a pelvic floor muscle tracking system 1, according to some examples, and also referred to below simply as “the system 1.”

The system 1 includes at least an intra-body device, such as the device 10 shown in FIG. 1. The device 10 is thus used as a non-limiting example of an intra-body device of the system 1 below.

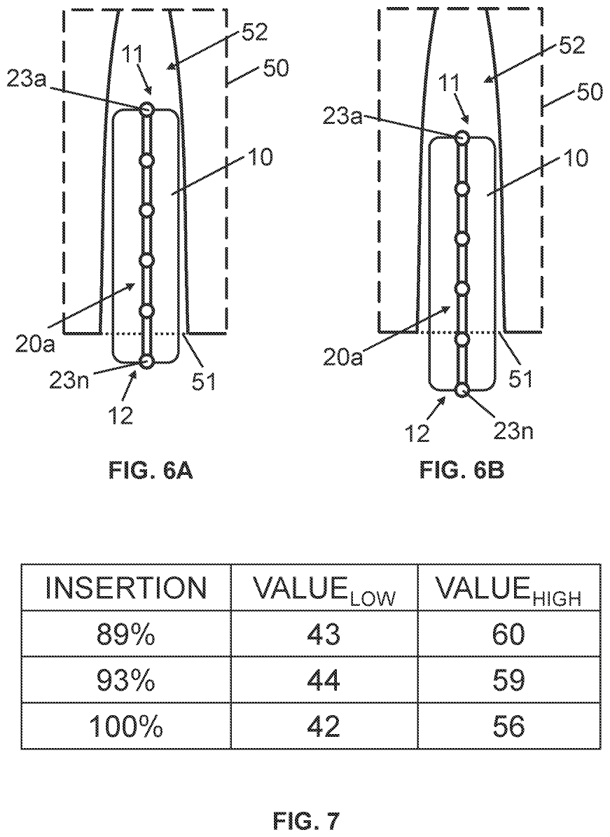

As shown in FIG. 2, the device 10 includes at least one pressure sensor 13 and at least one capacitance sensor 20. Further, the device 10 can optionally include at least one motion sensor 14 (as indicated with dashed borders). As aforesaid the device 10 includes, in some examples, at least one memory 15 and/or a data communications module 16, such as a wireless communications module (both also indicated with dashed borders). The device 10 includes, in some examples, at least one processor 17 (also indicated with dashed borders) for processing measurements of, for example, one or more of the at least one capacitance sensor 20, the at least one pressure sensor 13, and the at least one motion sensor 14. The at least one processor 17 can perform processing operations or facilitate determination of values such as the operations or values described with reference to FIGS. 5, 6A, 6B, and/or 7.

The system 1 also includes, in some examples, at least one computing or data processing device 30 as shown with dashed borders. The device 30 includes at least one processor 31, at least one memory 32, and a data communication module 33, such as a wireless communications module for providing data to and/or receiving data from the intra-body device 10. The at least one computing or data processing device 30 may conduct processing of measurements provided by sensors (e.g., one or more of the at least one capacitance sensor 20, the at least one pressure sensor 13, and the at least one motion sensor 14) of the intra-body device 10, and perform processing operations or facilitate determination of values such as the operations or values described with reference to FIGS. 5, 6A, 6B, and/or 7.

Additionally, or alternatively, the system 1 includes, in some examples, at least one apparatus 40 for communicating with the user. For example, the apparatus 40 can be for notifying or informing users about certain information or events. The at least one apparatus 40 is or comprises, for example, a sound-emitting device and/or a vibrator and/or a light-emitting device and/or a display. It may also include a data communications module such as a wireless communications module. The at least one apparatus 40 may be part of the intra-body device 10, part of the computing or data processing device 30, or be a separate apparatus (e.g., a mobile device of the user, such as a tablet or mobile phone).

The data communications modules 16, 33 may be adapted to allow wireless data exchange via a local area network, cellular network, or the like, and/or to allow wired data exchange.

FIG. 3 shows a capacitance sensor 20a for intra-body devices, according to some examples. The at least one capacitance sensor 20a may include a single capacitance sensor 20a with a plurality of sensing points 23a-23n, or a plurality of individual sensors 23a-23n, thereby making it possible to detect capacitance at several locations of the capacitance sensor 20a. The capacitance sensor 20a is arranged in linear manner, meaning that when projected onto a plane, it has the sensing points or sensors 23a-23n aligned or substantially aligned along one dimension as it can be observed in FIG. 3.

Each sensing point or sensor 23a-23n measures and provides its respective measured value V₁, V₂, . . . , Vₙ. Each measured value is measured at a specific time instant, therefore a plurality of measured values per sensing point or sensor 23a-23n can be provided for a plurality of time instants. In such cases, a time evolution of measurements per sensing point or sensor 23a-23n can be considered for processing such as that described with reference to FIGS. 5, 6A, 6B, and/or 7.

In at least some cases, the number of sensing points or sensors 23a-23n influences the precision with which it will be possible to determine the positioning of the intra-body device 10 inside the cavity. In this example, six sensing points or sensors 23a-23n have been represented in the capacitance sensor 20a for the sake of clarity and illustration only. It is, however, noted that in other examples there are more or fewer sensing points or sensors (e.g., ten, fifty, or five points or sensors).

FIG. 4 is a diagrammatic illustration of a capacitance sensor 20b for intra-body devices according to some examples. Like the capacitance sensor 20a of the examples of FIG. 3, the capacitance sensor 20b of the examples of FIG. 4 may include a single capacitance sensor 20a with a plurality of sensing points 23a-23n or a plurality of individual sensors 23a-23n. In contrast, the capacitance sensor 20b is arranged in matrix form, meaning that when projected onto a plane, it has the sensing points or sensors 23a-23n aligned or substantially aligned in two dimensions as it can be observed in FIG. 4.

In some examples, the two-dimensional arrangement allows the measurement of the capacitance not only along, for example, the length of the intra-body device 10, but also along, for example, a width dimension of the intra-body device 10. With the monitoring of the capacitance of a larger surface of the intra-body device 10, more accurate determinations of the placement of the device 10 within a cavity may be achievable, such as when determining if a portion of the device 10 is contacting one or more walls delimiting a cavity. Even though not illustrated, it is noted that each sensing point or sensor of the capacitance sensor measures and provides capacitance values according to its respective location.

As with the examples represented in FIG. 3, the number of sensing points or sensors 23 is adjusted in other examples. The number of sensing points or sensors 23 of the capacitance sensor 20b can be adjusted both in terms of both rows and columns, such as more or less than six rows and/or more or less than three columns for different precision in the determination to be made.

FIG. 5 shows an intra-body device 10 according to some examples, while partially inserted inside a cavity 52 of a user 50. For the sake of the illustration only, part of the cavity 52 of the user 50 has been represented together with an opening 51 that defines the cavity 52 and enables insertion of the intra-body device 10. Inner walls 53, such as pelvic floor muscles, delimit the cavity 52. The cavity 52 is, for example, a vaginal canal or an anal canal of the user 50.

The intra-body device 10 is represented as including at least one capacitance sensor 20a such as one of the examples described with reference to FIG. 3, but it will be noted that other capacitance sensors such as, but without limitation, the capacitance sensor 20b of the examples described with reference to FIG. 4 are possible as well.

The intra-body device 10 has part thereof within the cavity 52 in the position shown in FIG. 5, while some other part is outside the cavity 52 (e.g., not having passed through the opening 51).

In this example, measurements of all sensing points or sensors 23a-23f is considered, with the first sensing point or sensor 23a being closer to the first end 11 of the device 10 than a last sensing point or sensor 23f, which in turn is closer to the second end 12 of the device 10 than the first sensing point or sensor 23a.

By processing the measurements of the at least one capacitance sensor 20a, either the intra-body device 10 or a computing or data processing device (e.g., a component of the system 1 of FIG. 2) is capable of determining whether the intra-body device 10 is at least partially introduced in the cavity 52. What is more, in some examples, by considering the measurements of some or all sensing points or sensors 23a-23f, the intra-body device 10 or the computing or data processing device (e.g., of the system 1) is capable of determining the level of introduction of the intra-body device within the cavity 52.

By way of example, in the position shown in FIG. 5, the capacitance measured by first and second sensing points or sensors 23a-23b has values different than those of the capacitance measured by third, fourth, fifth, and sixth sensing points or sensors 23c-23f. Through a comparison of the measured values corresponding to each of the first and second sensing points or sensors 23a-23b with the measured values corresponding to each of the third, fourth, fifth, and sixth sensing points or sensors 23c-23f or a predetermined threshold value (such as a predetermined minimum capacitance value), it can be determined (e.g., by the device 10 or the system 1) that at least two sixths of the capacitance sensor 20a is introduced in the cavity 52. The comparison can likewise be the other way around, that is to say, of the measured values corresponding to each of the third, fourth, fifth, and sixth sensing points or sensors 23c-23f with the measured values corresponding to each of the first and second sensing points or sensors 23a-23b or a predetermined threshold value (such as a predetermined maximum capacitance value).

By having defined the arrangement of the at least one capacitance sensor 20a on the intra-body device 10 in terms of positioning relative to the extension of the latter, and the length of the at least one capacitance sensor 20a, it can be established (e.g., by the device 10 or the system 1) what percentage of the length of the intra-body device 10 is within the cavity 52. In this case, since the first end 21 of the capacitance sensor 20a is at, approximately, a distance of the first end 11 of the device 10 that is a 15% of the length of the intra-body device 10, and since the second end 22 of the capacitance sensor 20a is at the second end 12 of the device 10, the introduced two sixths of the capacitance sensor 20a corresponds to a level of introduction of the intra-body device 10 of approximately 48%.

The determination of at least partial insertion of the intra-body device 10 is made (e.g., by the device 10 or the system 1) when at least some sensing points or sensors 23a-23f are detected to be within the cavity 52 of the user 50, therefore when the capacitance measured value(s) of one or more sensing points or sensors 23a-23f is as explained above.

The level of introduction may be used (e.g., by the system 1) to establish whether the intra-body device is adequately introduced in the cavity 52. To this end, for example, a predetermined minimum insertion threshold can be defined for comparison with the obtained level of introduction. Any determined level of introduction below such threshold will be considered (e.g., by the system 1) as inadequate introduction of the intra-body device 10, and as adequate introduction otherwise.

Examples described herein may thus address or alleviate technical challenges associated with determining whether an intra-body device is properly positioned.

FIG. 6A and FIG. 6B show an intra-body device 10, according to some examples. Specifically, FIG. 6A shows the intra-body device 10 at a first time instant, and FIG. 6B shows the device 10 at a second time instant that is posterior to the first time instant.

In these examples, a first end 21 of the at least one capacitance sensor 20a is at a first end 11 of the device 10, and a second end 22 of the at least one capacitance sensor 20b is at a second end 12 of the device 10. In other examples, a different arrangement is provided, for example, the first end 21 of the sensor(s) 20a does not reach the first end 11 of the device 10, and/or the second end 22 of the sensor(s) does not reach the second end 12 of the device 10.

At the first time instant as depicted in FIG. 6A, the intra-body device 10 is at a first position within the cavity 52. At the second time instant as depicted in FIG. 6B, the intra-body device 10 is at a second position within the cavity 52. That is to say, the device 10 exhibits outwards motion from the first time instant to the second time instant. The change in position may be due, for example, to the motion forced to the intra-body device 10 as a result of the elongation, contraction or change of shape of the muscles of the pelvic floor of the user 50. The outwards motion can be associated with pressure exerted by the user 50 on the pelvic floor, like when the user 50 is performing a rehabilitation session. As explained, the device 10 obtains measured capacitance values corresponding to each sensing point or sensor 23a-23n.

The system 1 or the device 10 itself can, for example, process the measured capacitance values to make one or more determinations. In this case, the measured capacitance values are indicative of the change in position, in this case the outwards motion. For example, the system 1 or the device 10 can process the values as described and determine an elongation of the pelvic floor muscles. When the outwards motion is quantified, which means computing how much distance the intra-body device 10 has moved along the cavity 52, the level of elongation may also be estimated by the device 10 or the system 1. In examples where the intra-body device 10 includes a motion sensor, such as an accelerometer or an Inertial Measurement Unit, the measured capacitance values and the measured motion can be processed by the device 10 or system 1 in combined form to yield more accurate motion determinations.

One possible quantification of the motion involves determining the level of introduction of the device 10 in the cavity 52 at the first time instant and at the second time instant. The process can be repeated for each or any pair of time instants at which the at least one capacitance sensor 20a measured capacitance, either consecutive time instants or non-consecutive time instants. When the time instants are consecutive, the change in level of introduction may be divided by the sampling period of the capacitance sensor 20a to find out the instantaneous change in position. When multiple computations of this type are repeated over time, the complete motion of the device 10 may be quantified upon finding the time instants when the instantaneous change in position becomes zero or changes sign, meaning that the user 50 stopped the motion in one direction or ended the motion. Further, in some examples, by concatenating such processing, the motion sequence corresponding to outwards and inwards motion of the device 10 may be obtained. A motion sequence can be useful to improve the accuracy of a pelvic floor muscle tracking sequence. For example, the system 1 can cause display of the motion sequence or perform processing to determine user performance.

Alternatively, a similar processing can be performed, in some other examples, by considering, as first time instant, that when the intra-body device 10 has the greatest level of introduction in the cavity 52 within a specific time window and, as second time instant, that when the intra-body device 10 has the lowest level of introduction in the cavity 52 within the same specific time window. The difference in level of introduction defines the amount of outwards motion. By reversing the greatest and lowest level of introductions sequentially, the motion sequence can be obtained.

Pelvic floor muscle tracking sequences, which are defined by pressure measurements, include information about the performance of elongation and contraction of the muscles. A motion sequence, if there is any provided, can assist in the provision of a pelvic floor muscle tracking sequence since the movement can be at least partially associated with elongation and contraction.

FIG. 7 shows an exemplary table of a predetermined set of calibration values, according to some examples. The table of FIG. 7 can be used for calibration of pelvic floor muscle tracking whereby pressure values provided by at least one pressure sensor (e.g., the pressure sensor 13) of an intra-body device (e.g., the device 10) are adjusted based on the level of introduction of the device in a cavity of a user.

The values of the table are obtainable upon performing a calibration routine. Said routine involves the user exerting some pressure in the pelvic floor muscles. During that time, pressure and capacitance measurements are provided as described elsewhere herein, for example. The level of introduction of the device within the cavity is quantifiable in the manners previously described. The quantified level of introduction is then associated with the measured pressure values when the user exerts the specific pressure(s) on the pelvic floor muscles. The specific pressure(s) may include lower and higher values, e.g., minimum and maximum pressure exerted by the user, so that said limit values are considered for the calibration.

Referring to the table of FIG. 7 itself, it can be appreciated that the set of calibration values includes exemplary low and high pressures (in no specific units for the sake of clarity and illustration only) for levels of introduction of the intra-body device of 89%, 93% and 100% (full introduction). It is noted that more or fewer calibration values, and corresponding to other levels of introduction and minimum and high values are possible.

When pelvic floor muscle tracking is to be calibrated, in some cases the level of introduction of the intra-body device is determined at different time instants during a rehabilitation or exercise routine for instance. The pressure value(s) corresponding to those time instants are to be calibrated based on the determined level of introduction. For example, when the determined level of introduction is 95%, the low or high values corresponding to the level of introduction of 93% are applied for calibration. Selecting one or other level of introduction can be set beforehand according to some specific criterion. Furthermore, selecting the low value or the high value can be made dependent upon the type of effort that is determined, for example, whether it is elongation or contraction of the pelvic floor muscles.

Examples described herein may thus address or alleviate technical challenges associated with ensuring that an intra-body device is calibrated to an actual position thereof among a range of positions within the cavity, and thereby ensuring that measurements taken more accurately represent the activity of the pelvic floor.

Although specific examples are described herein, it will be evident that various modifications and changes may be made to these examples without departing from the broader spirit and scope of the disclosure. Accordingly, the specification and drawings are to be regarded in an illustrative rather than a restrictive sense. The accompanying drawings that form a part hereof show by way of illustration, and not of limitation, specific examples in which the subject matter may be practiced. The examples illustrated are described in sufficient detail to enable those skilled in the art to practice the teachings disclosed herein. Other examples may be utilized and derived therefrom, such that structural and logical substitutions and changes may be made without departing from the scope of this disclosure. This detailed description, therefore, is not to be taken in a limiting sense, and the scope of various examples is defined only by the appended claims, along with the full range of equivalents to which such claims are entitled.

Such examples of the subject matter may be referred to herein, individually or collectively, by the term “example” merely for convenience and without intending to voluntarily limit the scope of this application to any single example or concept if more than one is in fact disclosed. Thus, although specific examples have been illustrated and described herein, it should be appreciated that any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

Some portions of the subject matter discussed herein may be presented in terms of algorithms or symbolic representations of operations on data stored as bits or binary digital signals within a machine memory (e.g., a computer memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” and “an” are herein used, as is common in patent documents, to include one or more than one instance. As used herein, the conjunction “or” refers to a non-exclusive “or,” unless specifically stated otherwise.

Unless the context clearly requires otherwise, throughout the description and the claims, the words “comprise,” “comprising,” “include,” “including,” and the like are to be construed in an inclusive sense, as opposed to an exclusive or exhaustive sense, e.g., in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words using the singular or plural number may also include the plural or singular number, respectively. The word “or” in reference to a list of two or more items, covers all of the following interpretations of the word: any one of the items in the list, all of the items in the list, and any combination of the items in the list.

In the present disclosure the terms “first”, “second”, etc. have been used herein to describe certain elements, devices, or parameters, but it will be understood that (unless the context clearly indicates otherwise) the elements, devices, or parameters should not be limited by these terms since the terms are only or primarily used to distinguish one element, device, or parameter from another.

Although some examples may include a particular sequence of operations, the sequence may in some cases be altered without departing from the scope of the present disclosure. For example, some of the operations depicted may be performed in parallel or in a different sequence that does not materially affect the functions as described in the examples. In other examples, different components of an example device or system that implements an example method may perform functions at substantially the same time or in a specific sequence.

As used herein, the term “processor” may refer to any one or more circuits or virtual circuits (e.g., a physical circuit emulated by logic executing on an actual processor) that manipulates data values according to control signals (e.g., commands, opcodes, machine code, control words, macroinstructions, etc.) and which produces corresponding output signals that are applied to operate a machine. A processor may, for example, include at least one of a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), a Tensor Processing Unit (TPU), a Neural Processing Unit (NPU), a Vision Processing Unit (VPU), a Machine Learning Accelerator, an Artificial Intelligence Accelerator, an Application Specific Integrated Circuit (ASIC), a Field Programmable Gate Array (FPGA), a Radio-Frequency Integrated Circuit (RFIC), a Neuromorphic Processor, a Quantum Processor, or any combination thereof. A processor may be a multi-core processor having two or more independent processors (sometimes referred to as “cores”) that may execute instructions contemporaneously. Multi-core processors may contain multiple computational cores on a single integrated circuit die, each of which can independently execute program instructions in parallel. Parallel processing on multi-core processors may be implemented via architectures like superscalar, VLIW, vector processing, or SIMD that allow each core to run separate instruction streams concurrently. A processor may be emulated in software, running on a physical processor, as a virtual processor or virtual circuit. The virtual processor may behave like an independent processor but is implemented in software rather than hardware.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules/components that operate to perform one or more operations or functions. The modules/components referred to herein may, in some examples, comprise processor-implemented modules/components.

Similarly, the methods described herein may be at least partially processor-implemented. For example, at least some of the operations of a method may be performed by one or more processors or processor-implemented modules/components. The performance of certain of the operations may be distributed among the one or more processors, not only residing within a single machine, but deployed across a number of machines. In some examples, the processor or processors may be located in a single location (e.g., within a home environment, an office environment, or a server farm), while in other examples the processors may be distributed across a number of locations.

Examples may be implemented in digital electronic circuitry, or in computer hardware, firmware, or software, or in combinations of them. Examples may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

In view of the above-described implementations of subject matter this application discloses the following list of examples, wherein one feature of an example in isolation or more than one feature of an example, taken in combination and, optionally, in combination with one or more features of one or more further examples are further examples also falling within the disclosure of this application.

Example 1 is a computer-implemented method comprising: accessing, by at least one processor, capacitance measurements taken by at least one capacitance sensor of an intra-body device, the at least one capacitance sensor being arranged such that the at least one capacitance sensor extends along at least part of a length of the intra-body device; processing, by the at least one processor, the capacitance measurements to determine positioning of the intra-body device in relation to a cavity of a body of a user, the cavity being at least one of a vagina or an anus; and controlling, by the at least one processor, operation of a pelvic floor muscle tracking system based on the determined positioning.

In Example 2, the subject matter of Example 1 includes, wherein the determined positioning comprises a level of introduction of the intra-body device within the cavity.

In Example 3, the subject matter of Example 2 includes, wherein the level of introduction indicates that the intra-body device is fully introduced in the cavity, partially introduced in the cavity, or not introduced in the cavity.

In Example 4, the subject matter of any of Examples 1-3 includes, wherein the determining of the positioning of the intra-body device comprises determining, by the at least one processor and based on the capacitance measurements, that at least a first portion of the intra-body device is contacting one or more walls delimiting the cavity, the first portion comprising at least part of the at least one capacitance sensor.

In Example 5, the subject matter of Example 4 includes, wherein the at least one capacitance sensor is arranged externally on a surface of the intra-body device.

In Example 6, the subject matter of any of Examples 1-5 includes, wherein the processing of the capacitance measurements comprises comparing at least a subset of the capacitance measurements to one or more predetermined thresholds.

In Example 7, the subject matter of any of Examples 1-6 includes, processing, by the at least one processor, exercise measurements taken by at least one pressure sensor of the intra-body device, wherein the exercise measurements are taken while the user is doing one or more exercises with the intra-body device introduced in the cavity; performing, by the at least one processor and based on the processed exercise measurements, pelvic floor muscle tracking to track contraction or elongation of one or more muscles of a pelvic floor of the user; and calibrating, by the at least one processor, the pelvic floor muscle tracking by using predetermined calibration values that are associated with positioning of the intra-body device relative to the cavity.

In Example 8, the subject matter of Example 7 includes, wherein the determining of the positioning of the intra-body device comprises determining, by the at least one processor and based on the capacitance measurements, a level of introduction of the intra-body device within the cavity, and wherein the predetermined calibration values relate at least one predetermined level of introduction of the intra-body device to at least one predetermined pressure value.

In Example 9, the subject matter of any of Examples 7-8 includes, accessing, by the at least one processor, calibration measurements taken by the at least one pressure sensor of the intra-body device, wherein the calibration measurements are taken while the user is conducting a calibration of the intra-body device with the intra-body device introduced in the cavity, and wherein the calibration measurements and the predetermined calibration values are used to calibrate the pelvic floor muscle tracking.

In Example 10, the subject matter of Example 9 includes, wherein the determining of the positioning of the intra-body device comprises determining, by the at least one processor and based on the capacitance measurements, one or more levels of introduction of the intra-body device within the cavity, and wherein the predetermined calibration values relate each determined level of introduction of the intra-body device to at least one pressure value of the calibration measurements taken at the determined level of introduction.

In Example 11, the subject matter of any of Examples 1-10 includes, wherein the controlling of the operation of the pelvic floor muscle tracking system comprises causing transmission of a notification indicative of a calibration operation of the intra-body device to be conducted by the user.

In Example 12, the subject matter of any of Examples 1-11 includes, wherein the determined positioning comprises a level of introduction of the intra-body device within the cavity, the level of introduction indicating that the intra-body device is partially introduced in the cavity or not introduced in the cavity, and wherein the controlling of the operation of the pelvic floor muscle tracking system comprises causing, based on the determined positioning, transmission of a notification to the user indicative of the determined positioning.

In Example 13, the subject matter of any of Examples 1-12 includes, wherein the processing of the capacitance measurements to determine the positioning of the intra-body device comprises determining, by the at least one processor and based on the capacitance measurements, whether the intra-body device moves inwards or outwards in the cavity, and wherein the controlling of the operation of the pelvic floor muscle tracking system comprises providing, based on the moving of the intra-body device, a pelvic floor motion tracking sequence indicative of contraction or elongation of one or more muscles of a pelvic floor of the user.

In Example 14, the subject matter of any of Examples 1-13 includes, wherein the determined positioning comprises a level of introduction of the intra-body device within the cavity, the level of introduction indicating that the intra-body device is partially introduced in the cavity or not introduced in the cavity, and wherein the controlling of the operation of the pelvic floor muscle tracking system comprising halting pelvic floor muscle tracking based on identifying that the intra-body device is partially introduced in the cavity or not introduced in the cavity.

In Example 15, the subject matter of any of Examples 1-14 includes, wherein the at least one capacitance sensor comprises at least one of: a plurality of sensing positions in a linear or matrix arrangement; or a plurality of capacitance sensors in a linear or matrix arrangement.

In Example 16, the subject matter of any of Examples 1-15 includes, wherein the at least one capacitance sensor is arranged such that at least a portion thereof is at or near a first end of the intra-body device, the first end being opposite a second end of the intra-body device, and the second end corresponding to an introduction end of the intra-body device in the cavity.

Example 17 is an intra-body device comprising a body portion to be introduced in a cavity of a body of a user, the cavity being at least one of a vagina or an anus, and the body portion comprising: at least one pressure sensor; and at least one capacitance sensor extending along at least part of a length of the body portion.

In Example 18, the subject matter of Example 17 includes, wherein the at least one capacitance sensor comprises at least one of: a plurality of sensing positions in a linear or matrix arrangement; or a plurality of capacitance sensors in a linear or matrix arrangement.

In Example 19, the subject matter of any of Examples 17-18 includes, wherein the at least one capacitance sensor is arranged such that at least a portion thereof is at or near a first end of the body portion, the first end being opposite a second end of the body portion, and the second end corresponding to an introduction end of the intra-body device in the cavity.

Example 20 is a pelvic floor muscle tracking system comprising: at least one processor; and memory storing instructions that, when executed by the at least one processor, configure the at least one processor to perform operations comprising: accessing capacitance measurements taken by at least one capacitance sensor of an intra-body device, the at least one capacitance sensor being arranged such that the at least one capacitance sensor extends along at least part of a length of the intra-body device; processing the capacitance measurements to determine positioning of the intra-body device in relation to a cavity of a body of a user, the cavity being at least one of a vagina or an anus; and controlling operation of the pelvic floor muscle tracking system based on the determined positioning.

Example 21 is at least one machine-readable medium including instructions that, when executed by processing circuitry, cause the processing circuitry to perform operations to implement any of Examples 1-20.

Example 22 is an apparatus comprising means to implement any of Examples 1-20.

Example 23 is a system to implement any of Examples 1-20.

Example 24 is a method to implement any of Examples 1-20.

What is claimed is:

1. A computer-implemented method comprising:
accessing, by at least one processor, capacitance measurements taken by at least one capacitance sensor of an intra-body device, the at least one capacitance sensor being arranged such that the at least one capacitance sensor extends along at least part of a length of the intra-body device;
processing, by the at least one processor, the capacitance measurements to determine positioning of the intra-body device in relation to a cavity of a body of a user, the cavity being at least one of a vagina or an anus; and
controlling, by the at least one processor, operation of a pelvic floor muscle tracking system based on the determined positioning.

2. The method of claim 1, wherein the determined positioning comprises a level of introduction of the intra-body device within the cavity.

3. The method of claim 2, wherein the level of introduction indicates that the intra-body device is fully introduced in the cavity, partially introduced in the cavity, or not introduced in the cavity.

4. The method of claim 1, wherein the determining of the positioning of the intra-body device comprises determining, by the at least one processor and based on the capacitance measurements, that at least a first portion of the intra-body device is contacting one or more walls delimiting the cavity, the first portion comprising at least part of the at least one capacitance sensor.

5. The method of claim 4, wherein the at least one capacitance sensor is arranged externally on a surface of the intra-body device.

6. The method of claim 1, wherein the processing of the capacitance measurements comprises comparing at least a subset of the capacitance measurements to one or more predetermined thresholds.

7. The method of claim 1, further comprising:
processing, by the at least one processor, exercise measurements taken by at least one pressure sensor of the intra-body device, wherein the exercise measurements are taken while the user is doing one or more exercises with the intra-body device introduced in the cavity;
performing, by the at least one processor and based on the processed exercise measurements, pelvic floor muscle tracking to track contraction or elongation of one or more muscles of a pelvic floor of the user; and
calibrating, by the at least one processor, the pelvic floor muscle tracking by using predetermined calibration values that are associated with positioning of the intra-body device relative to the cavity.

8. The method of claim 7, wherein the determining of the positioning of the intra-body device comprises determining, by the at least one processor and based on the capacitance measurements, a level of introduction of the intra-body device within the cavity, and wherein the predetermined calibration values relate at least one predetermined level of introduction of the intra-body device to at least one predetermined pressure value.

9. The method of claim 7, further comprising:
accessing, by the at least one processor, calibration measurements taken by the at least one pressure sensor of the intra-body device, wherein the calibration measurements are taken while the user is conducting a calibration of the intra-body device with the intra-body device introduced in the cavity, and wherein the calibration measurements and the predetermined calibration values are used to calibrate the pelvic floor muscle tracking.

10. The method of claim 9, wherein the determining of the positioning of the intra-body device comprises determining, by the at least one processor and based on the capacitance measurements, one or more levels of introduction of the intra-body device within the cavity, and wherein the predetermined calibration values relate each determined level of introduction of the intra-body device to at least one pressure value of the calibration measurements taken at the determined level of introduction.

11. The method of claim 1, wherein the controlling of the operation of the pelvic floor muscle tracking system comprises causing transmission of a notification indicative of a calibration operation of the intra-body device to be conducted by the user.

12. The method of claim 1, wherein the determined positioning comprises a level of introduction of the intra-body device within the cavity, the level of introduction indicating that the intra-body device is partially introduced in the cavity or not introduced in the cavity, and wherein the controlling of the operation of the pelvic floor muscle tracking system comprises causing, based on the determined positioning, transmission of a notification to the user indicative of the determined positioning.

13. The method of claim 1, wherein the processing of the capacitance measurements to determine the positioning of the intra-body device comprises determining, by the at least one processor and based on the capacitance measurements, whether the intra-body device moves inwards or outwards in the cavity, and wherein the controlling of the operation of the pelvic floor muscle tracking system comprises providing, based on the moving of the intra-body device, a pelvic floor motion tracking sequence indicative of contraction or elongation of one or more muscles of a pelvic floor of the user.

14. The method of claim 1, wherein the determined positioning comprises a level of introduction of the intra-body device within the cavity, the level of introduction indicating that the intra-body device is partially introduced in the cavity or not introduced in the cavity, and wherein the controlling of the operation of the pelvic floor muscle tracking system comprising halting pelvic floor muscle tracking based on identifying that the intra-body device is partially introduced in the cavity or not introduced in the cavity.

15. The method of claim 1, wherein the at least one capacitance sensor comprises at least one of: a plurality of sensing positions in a linear or matrix arrangement; or a plurality of capacitance sensors in a linear or matrix arrangement.

16. The method of claim 1, wherein the at least one capacitance sensor is arranged such that at least a portion thereof is at or near a first end of the intra-body device, the first end being opposite a second end of the intra-body device, and the second end corresponding to an introduction end of the intra-body device in the cavity.

17. An intra-body device comprising a body portion to be introduced in a cavity of a body of a user, the cavity being at least one of a vagina or an anus, and the body portion comprising:
at least one pressure sensor; and
at least one capacitance sensor extending along at least part of a length of the body portion.

18. The intra-body device of claim 17, wherein the at least one capacitance sensor comprises at least one of: a plurality of sensing positions in a linear or matrix arrangement; or a plurality of capacitance sensors in a linear or matrix arrangement.

19. The intra-body device of claim 17, wherein the at least one capacitance sensor is arranged such that at least a portion thereof is at or near a first end of the body portion, the first end being opposite a second end of the body portion, and the second end corresponding to an introduction end of the intra-body device in the cavity.

20. A pelvic floor muscle tracking system comprising:
at least one processor; and
memory storing instructions that, when executed by the at least one processor, configure the at least one processor to perform operations comprising:
accessing capacitance measurements taken by at least one capacitance sensor of an intra-body device, the at least one capacitance sensor being arranged such that the at least one capacitance sensor extends along at least part of a length of the intra-body device;
processing the capacitance measurements to determine positioning of the intra-body device in relation to a cavity of a body of a user, the cavity being at least one of a vagina or an anus; and
controlling operation of the pelvic floor muscle tracking system based on the determined positioning.
